(19) United States
(12) Patent Application Publication
(10) Pub. No.: US 2023/0097655 A1
(43) Pub. Date: Mar. 30, 2023

(54) APPARATUS FOR GENERATING MICROFLUIDIC CONCENTRATION FIELD, METHOD OF FABRICATING THE APPARATUS FOR GENERATING MICROFLUIDIC CONCENTRATION FIELD AND APPARATUS FOR FLUID FLOW

(71) Applicant: UNIST(ULSAN NATIONAL INSTITUTE OF SCIENCE AND TECHNOLOGY), Ulsan (KR)

(72) Inventors: Taesung Kim, Ulsan (KR); Juyeol Bae, Ulsan (KR)

(73) Assignee: UNIST(ULSAN NATIONAL INSTITUTE OF SCIENCE AND TECHNOLOGY), Ulsan (KR)

(21) Appl. No.: 17/947,205

(22) Filed: Sep. 19, 2022

(30) Foreign Application Priority Data
Sep. 24, 2021 (KR) 10-2021-0126356

(51) Int. Cl. B01L 3/00

(52) U.S. Cl. CPC B01L 3/502707; B01L 3/502753; B01L 2200/12; B01L 2200/0631

(57) ABSTRACT

Provided is an apparatus for generating a microfluidic concentration field, the apparatus including: a substrate; a base film disposed on the substrate; a microchannel, which is formed in a space between the substrate and the base film and through which a fluid flows; a through passage, which communicates with the microchannel and is configured to pass through the base film; and a membrane, which is formed at a portion where the microchannel and the through passage communicate with each other and allows the fluid flowing along the microchannel and the through passage or a material flowing together with the fluid to selectively pass through the membrane, wherein a concentration field is formed between the fluid of the through passage and the fluid of the microchannel by the membrane.

### CROSS-REFERENCE TO RELATED APPLICATION

This application claims the benefit of Korean Patent Application No. 10-2021-0126356, filed on Sep. 24, 2021, in the Korean Intellectual Property Office, the disclosure of which is incorporated herein in its entirety by reference.

### TECHNICAL FIELD

The present invention relates to an apparatus for generating a microfluidic concentration field, a method of fabricating the apparatus for generating a microfluidic concentration field, and an apparatus for fluid flow, and more particularly, to an apparatus for generating a microfluidic concentration field, whereby a pixelized concentration field may be generated by a membrane formed at a portion where a microchannel and a through passage communicate with each other, a method of fabricating the apparatus for generating a microfluid concentration field, and an apparatus for fluid flow.

### BACKGROUND ART

Microfluidic chips are chips including a microchannel and a chamber through which a fluid flows, are provided on a substrate fabricated of various materials such as plastic, glass or silicon. Various types of fluids, such as blood, body fluids, reagents, badges or cell culture mediums, can move through the microchannel, and the microfluidic chips are widely used in clinical diagnosis, bio field, medicine and fine chemistry fields. Microfluidic technology is applied on a single chip or substrate, which allows the entire research process performed in a laboratory to be integrated into a single chip. Microfluidic chips, such as lab-on chips, include complex dimensional configuration, such as a mixer, a fluid separation channel, a valve, and the like, so as to integrate various required functions. The use frequency of research on microfluids is gradually increasing in performing cell-based research and other applied researches. Because microfluidic-based research provides faster and more sensitive detection results while using a smaller volume of preparation, microfluidic-based research has several advantages over conventional laboratory-level analytical processes.

In this way, concentration gradients are widely involved in natural phenomena including colloidal transport. In order to precisely observe these concentration gradients, a microfluidic apparatus for generating a precise two-dimensional (2D) concentration field is required. However, because most of devices related art are based on a 2D microchannel network, a source/sink is disposed only on a sidewall of a 2D field, and the versatility of generating various fields is insufficient.

### DISCLOSURE OF THE INVENTION

The present invention provides an apparatus for generating a microfluidic concentration field, whereby a pixelized concentration field may be generated by a membrane formed at a portion where a microchannel and a through passage communicate with each other, a method of fabricating the apparatus for generating the microfluidic concentration field, and an apparatus for fluid flow.

According to an aspect of the present invention, there is provided an apparatus for generating a microfluidic concentration field, the apparatus including a substrate, a base film disposed on the substrate, a microchannel, which is formed in a space between the substrate and the base film and through which a fluid flows, a through passage, which communicates with the microchannel and is configured to pass through the base film, and a membrane, which is formed at a portion where the microchannel and the through passage communicate with each other and allows the fluid flowing along the microchannel and the through passage or a material flowing together with the fluid to selectively pass through the membrane, wherein a concentration field is formed between the fluid of the through passage and the fluid of the microchannel by the membrane.

According to another aspect of the present invention, there is provided a method of fabricating an apparatus for generating a microfluidic concentration field, the method including preparing a microfluidic film and disposing the microfluidic film on a substrate, the microfluidic film including a base film, a microchannel, which is formed on the base film and through which a fluid flows and a through passage communicating with the microfluidic channel and being configured to pass through the base film, and forming a membrane, the membrane being formed at a portion where the microchannel and the through passage communicate with each other and allowing the fluid flowing along the microchannel and the through passage to selectively pass through the membrane.

According to another aspect of the present invention, there is provided an apparatus for fluid flow, the apparatus including a substrate, a base film disposed on the substrate, a microchannel, which is defined by a space between the substrate and the base film and through which a fluid flows, a through passage, which communicates with the microchannel and is configured to pass through the base film, and a membrane, which is formed at a portion where the microchannel and the through passage communicate with each other and allows the fluid flowing along the microchannel and the through passage or a material flowing together with the fluid to selectively pass through the membrane.

An apparatus for generating a microfluidic concentration field, a method of fabricating the apparatus for generating the microfluidic concentration field, and an apparatus for fluid flow according to the present invention have the following effects.

First, a pixelized concentration field can be generated by a membrane formed at a portion where a microchannel and a through passage communicate with each other.

Second, because the pixelized concentration field can be generated, several membranes are disposed on the entire plane of a substrate so that various concentration fields can be formed.

Third, because various concentration fields can be formed, rapid and various analyses on a flowing fluid are possible compared to a single concentration field.

### BRIEF DESCRIPTION OF THE DRAWINGS

FIG. 1 is a schematic diagram of an apparatus for generating a microfluidic concentration field according to an embodiment of the present invention;

FIG. 2 is a schematic diagram schematically illustrating a state in which a concentration field of the apparatus for generating the microfluidic concentration field shown in FIG. 1 is generated;

FIG. 3 is a schematic diagram illustrating a method of fabricating a microfluidic film of the apparatus for generating the microfluidic concentration field shown in FIG. 1;

FIG. 4 is a schematic diagram and a photo illustrating a state in which the apparatus for generating the microfluidic concentration field shown in FIG. 1 operates;

FIG. 5 is a photo showing respective states in which a concentration field is formed when the structure of the apparatus for generating the microfluidic concentration field shown in FIG. 1 is changed;

FIG. 6 is a photo showing various operation states of the apparatus for generating the microfluidic concentration field shown in FIG. 1;

FIG. 7 is a schematic diagram illustrating a state in which a selfassembled particle membrane (SAPM) is omitted from a microfluidic film included in the apparatus for generating the microfluidic concentration field shown in FIG. 1;

FIG. 8 is a block diagram illustrating a method of fabricating a microfluidic film shown in FIG. 7;

FIG. 9 is a schematic diagram illustrating an operation of fabricating a basic mold that is a template for fabricating a master mold of the method of fabricating the microfluidic film shown in FIG. 8;

FIG. 10 is a schematic diagram illustrating an operation of fabricating a master mold of the method of fabricating the microfluidic film shown in FIG. 8;

FIG. 11 is a schematic diagram illustrating a state in which the master mold fabricated shown in FIG. 10 is separated from the basic mold;

FIG. 12 is a schematic diagram illustrating a method of fabricating a microfluidic film by using the master mold of the method of fabricating the microfluidic film shown in FIG. 8;

FIG. 13 is a schematic diagram illustrating another method of fabricating a microfluidic film by using the master mold of the method of fabricating the microfluidic film shown in FIG. 8; and

FIG. 14 is a schematic diagram illustrating an operation of transferring the microfluidic film fabricated by the method of fabricating the microfluidic film shown in FIG. 8, to a rigid substrate.

### DETAILED DESCRIPTION FOR CARRYING OUT THE INVENTION

Hereinafter, the present invention will be described in detail with reference to the accompanying drawings, in which exemplary embodiments of the invention are shown.

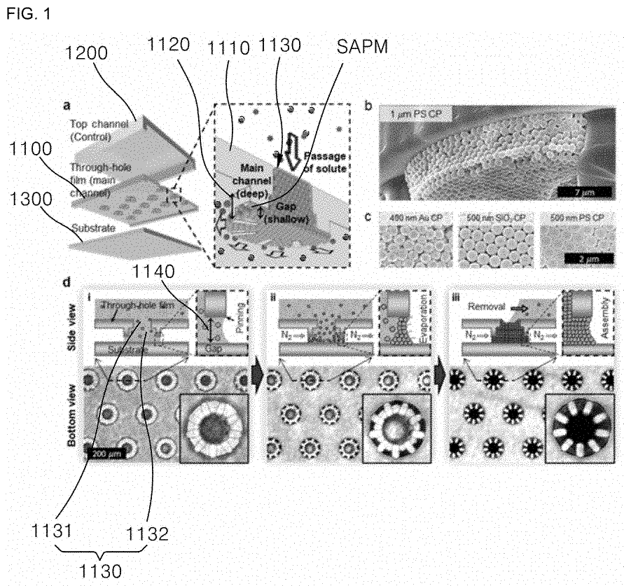

Referring to FIGS. 1 through 7, an apparatus 1000 for generating a microfluidic concentration field according to an embodiment of the present invention includes a microfluidic film 1100, a control film 1200, and a substrate 1300. In the present embodiment, the apparatus 1000 for generating the microfluidic concentration field includes all of the microfluidic film 1100, the control film 1200, and the substrate 1300. However, the apparatus 1000 for generating the microfluidic concentration field may include only the microfluidic film 1100 and the substrate 1300.

The microfluidic film 1100 is disposed on the substrate 1300. The microfluidic film 1100 includes a base film 1110, a microchannel 1120, a through passage 1130, a connection channel 1140, and a selfassembled particle membrane (SAPM). In this case, the base film 110 is a portion that constitutes the appearance (framework) of the microfluidic film 1000. In other words, the microfluidic film 1000 has a structure in which the microchannel 1120, the through passage 1130 and the connection channel 1140 are formed on the base film 1110. The base film 1110 is formed of resin. In detail, the base film 1110 is formed of an Off-stoichiometry thiol-ene polymers (OSTEmer) resin, but any material of the base film 1110 may be changed.

The microchannel 1120 is formed on the base film 1110 so that a fluid may flow through the microchannel 120. In detail, the microchannel 1120 is formed between the base film 1110 and the substrate 1200. The microchannel 1120 is formed on the base film 1110 in a longitudinal direction. The microchannel 1120 is formed in the form of a groove on the base film 1110. The microchannel 1120 is a micro-scale or nano-scale channel. However, any size of the microchannel 1120 may be changed. The microchannel 1120 may be formed in a radial form along the circumference of the through passage 1130.

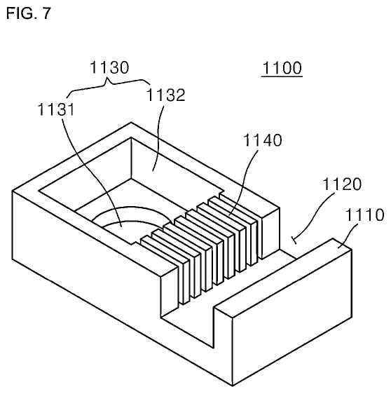

The through passage 1130 is formed to pass through the base film 1110. The through passage 1130 includes a first through passage and a second through passage, which are formed at an upper portion of the through passage 1130. The first through passage communicates with the control film 1200. The upper portion of the second through passage communicates with a lower portion of the first through passage and has a greater width than the width of the first through passage. The first through passage corresponds to the through passage lower hole 1131 of FIG. 7, and the second through passage corresponds to the through passage upper hole 1132 of FIG. 7.

The second through passage is open in the direction of the microchannel from a lower portion of a side surface to a portion spaced apart from the lower portion of the side surface by a set length in an upward direction. In the present embodiment, a cross-section of the open portion of the second through passage is circular. However, the present invention is not limited thereto, and any shape of the open portion may be changed. Also, in the present embodiment, the second through passage communicates with a plurality of microchannels formed in a direction crossing a direction in which the through passage is formed. In the present embodiment, a plurality of through passages 1130 are spaced apart from each other.

The through passage 1130 is formed to fluid-communicate with the control film 1200 stacked on the base film 1110. That is, the through passage 1130 is a passage on which the fluid does not flow only inside the base film 1110 but flows from the control film 1200 that is an outside of the base film 1110 to the through passage 1130. In the present embodiment, the through passage 1130 has a hole structure in which the through passage 1130 is spaced apart from the microchannel 1120 and passes through the base film 1110 from a top surface to a bottom surface of the base film 1110.

The through passage 1130 will be briefly described with reference to FIG. 7 again. The through passage 1130 includes a through passage lower hole 1131 and a through passage upper hole 1132. For reference, the microfluidic film 1100 in FIG. 7 is in a state in which, for convenience of illustration, the microfluidic film in FIG. 1 is turned upside down. Also, only portions of respective pixelized through passages 1130 formed in the microfluid film 1100 are illustrated. In particular, only portions of the microchannel 1120 and the connection channel 1140 that communicate with the through passage 1130 are illustrated.

Thus, in actuality, the through passage lower hole 1131 is disposed at an upper portion of the through passage upper hole 1132. The through passage lower hole 1131 is a portion that extends from the upper portion of the through passage 1130 downward by a set length. The through passage upper hole 1132 is a portion that communicates with the lower portion of the through passage lower hole 1131 and extends downward. In this case, in the present embodiment, the through passage upper hole 1132 has a greater width than that of the through passage lower hole 1131. Thus, a step height is formed between the through passage upper hole 1132 and the through passage lower hole 1131. In the present embodiment, the vertical length of the through passage upper hole 1132 is greater than the vertical length of the through passage lower hole 1131. In detail, the through passage upper hole 1132 has the vertical length of 60 μm, and the through passage lower hole 1131 has the vertical length of 20 μm.

The connection channel 1140 allows the microchannel 1120 and the through passage 1130 to communicate with each other. In the present embodiment, the connection channel 1140 is formed up to a portion (hereinafter, referred to as a ‘first region’) that extends from the top surface of the substrate 1300 upward by a set length. That is, the microchannel 1120 and the through passage 1130 communicate with each other through the first region that is a space extending from the substrate 1300 by a set length upward. One side of the connection channel 1140 communicates with the microchannel 1120, and the other side of the connection channel 1140 communicates with the through passage 1130. In the present embodiment, the connection channel 1140 is formed in a horizontal direction crossing the longitudinal direction in a two-dimensional plane of the base film 1110. In the present embodiment, a plurality of connection channel 1140 are spaced apart from each other in the longitudinal direction, but only one connection channel 1140 may be formed. Of course, the structure of the connection channel 1140 may be changed into any structure in which the microchannel 1120 and the through passage 1130 may communicate with each other. The connection channel 1140 is formed in the form of a groove on the base film 1110. The connection channel 1140 may be formed in a radial form along the circumference of the through passage 1130.

The SAPM is disposed on the through passage 1130 and the connection channel 1140. The SAPM allows the fluid flowing along the microchannel 1120 and the through passage 1130 or a material (e.g., ions, micro or nanoscale small particles) flowing together with the fluid to selectively pass through the SAPM. Through selective passage, a concentration field due to a concentration difference between the fluid of the through passage 1130 and the fluid of the microchannel 1120 is formed.

In the present embodiment, a plurality of through passages 1130 having a pixelized structure are formed on the microfluidic film 1100 to be spaced apart from each other. In FIG. 1, the pixel structure in which the through passage 1130 is formed, are arranged in a zigzag form. However, the pixelized structure may be in the form of a square or rectangular matrix.

The control film 1200 is stacked on the upper portion of the base film 1110 and communicates with the through passage 1130. In this case, the concentration field formed on the microchannel 1120 is controlled by the fluid flowing into the base film 1110 through a control channel (not shown) formed on the control film 1200 to communicate with the through passage 1130. In this case, the control channel is formed at a position corresponding to the through passage 1130 formed on the base film 1110. In the present embodiment, the control film 1200 is formed of an OSTEmer resin, but any material of the control film 1200 may be changed.

The substrate 1300 is a portion that constitutes bottom surfaces of the microchannel 1120, the through passage 1130 and the connection channel 1140. The substrate 1300 is formed of a synthetic resin having a flat plate. In the present embodiment, the substrate 1300 is formed of polydimethylsiloxane (PDMS). However, the present invention is not limited thereto, and the substrate 1300 may be formed of a film or glass. The apparatus 1000 for generating the microfluidic concentration field according to the present embodiment is to form a concentration field according to the flow of the microfluid. However, the apparatus 1000 for generating the microfluidic concentration field may be an apparatus for fluid flow.

The apparatus 1000 for generating the microfluidic concentration field may flow a fluid having a first concentration into the microchannel 1120 and may flow a fluid having a second concentration into a control channel of the control film 1200, thereby controlling the concentration field in a vertical direction. That is, by making the first concentration and the second concentration different, the fluid may flow from the microchannel 1200 to the control channel or from the control channel to the microchannel 1200. Thus, the apparatus 1000 for generating the microfluidic concentration field may generate a three-dimensional (3D) concentration field. In addition, a plurality of through passages 1130 and SAPMs are formed on the microfluidic film 1100 in a pixelized structure so that individual concentration field control in the entire microfluidic film 1100 is possible. At this time, microfluids having different concentrations may be injected into each pixelized structure. In this case, multiple different concentration field generation and control is possible at one time.

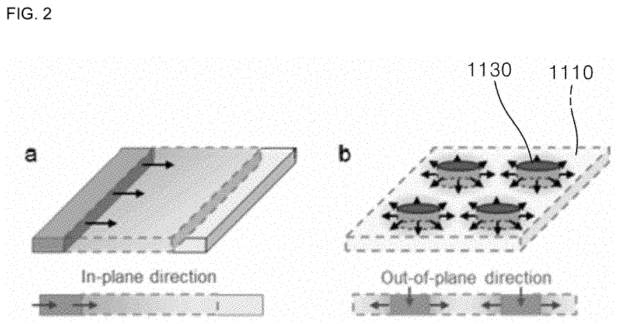

Referring to FIG. 2, a of FIG. 2 illustrates a state in which two main microchannels that exist on the same plane of a two-dimensional (2D) microchannel network according to the related art are connected to each other through a membrane. That is, in-plane connection through an SAPM causes generation of the entire concentration field from one sidewall to another sidewall of the concentration field. However, this method is insufficient to construct an individual concentration field in the entire plane of the 2D concentration field. On the other hand, b of FIG. 2 schematically illustrates the apparatus 1000 for generating a microfluidic concentration field according to the present embodiment, which shows that a plurality of concentration fields may be formed on the same plane in a pixelized manner.

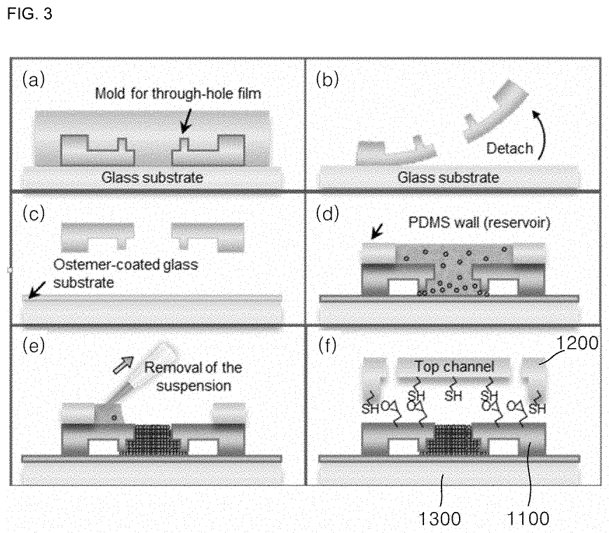

FIG. 3 schematically illustrates a method of fabricating the apparatus 1000 for generating the microfluidic concentration field. Two master molds having multi-layered patterns of a SU-8 photoresist are prepared on a silicon wafer by photolithography, and a PDMS duplicate is fabricated by a general soft-lithography process. In this case, one master mold (mold for fabricating the microfluidic film 1100 of FIG. 1) has a three-layer SU-8 pattern for a through-hole film, and another master mold (mold for fabricating the control film 1200 of FIG. 1) has a single-layer SU-8 pattern for an upper PDMS layer.

After PDMS treated with perfluorooctyltrimethoxysilane (PFOCTS) is put on glass, an Ostemer resin is loaded into a PDMS mold by vacuum driving flow and is cured with ultraviolet (UV) light (a). When an UV curing film is immersed into water, an Ostemer film may be easily detached from a glass substrate (b). A film is put on the glass substrate that is spin-coated with UV curing Ostemer (c). Then, the film is finally cured at an oven of 65° C. In order to integrate an SAPM into the prepared Ostemer film, a reservoir made of PDMS is put on the film (d). Next, the reservoir is filled with a silicon nanoparticle suspension. The suspension flows into a through-hole (corresponding to the through passage 1130 of FIG. 1) and a shallow gap (corresponding to the connection channel 1140 of FIG. 1) without overflowing into a main channel. This may be achieved through fine adjustment between a capillary force and liquid pinning by changing the mixture ratio of alcohol in the suspension and the degree of oxygen plasma treatment. When an N₂ gas flows through the main channel (corresponding to the microchannel 1120 of FIG. 1), particles are concentrated into the through-hole (corresponding to the through passage 1130 of FIG. 1) from the shallow gap (corresponding to the connection channel 1140 of FIG. 1). In order to reduce time consumption required to assemble the particles, the excessive suspension is removed (e). After the suspension is completely dried, the top surface of the film is cleaned with water. Then, upper PDMS (corresponding to the control film 1200 of FIG. 1) is prepared and stacked on the film (f).

First, a mold having a structure corresponding to the microchannel 1120, the through passage 1130 and the connection channel 1140 of FIG. 1 is fabricated. The fabricated mold is detached from the glass substrate and is disposed on the substrate 1300 of FIG. 1. Next, the suspension is injected into the through passage 1130 of FIG. 1, and a membrane is formed on the connection channel 1140. Next, by stacking the control film 1200 (Top channel), the apparatus 1000 for generating the microfluidic concentration field is fabricated.

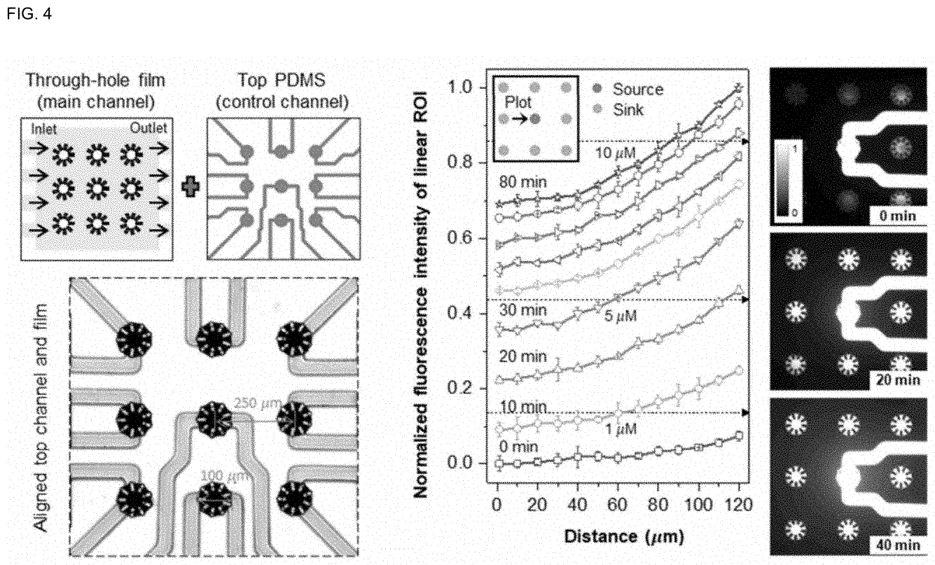

(a) of FIG. 4 illustrates a microfluidic source/sink array (MSA) apparatus based on a 3D microchannel having a 3×3 SAPM array that may be individually controlled in a square domain so as to investigate the generation of the 2D concentration field by using a selfassembled particle membrane array. In the drawing, Through-hole film refers to the microfluidic film 1100 of FIG. 1, and Top PDMS refers to the control film 1200 of FIG. 1. Through-hole film includes nine 80 μm height channels having additional inlet and outlet ports. A rectangular region of the main channel is an interest region for investigating diffusion phoresis colloidal transport in the gradient of a solute concentration. The device may be used to construct a desired chemical source/sink by continuously flowing new buffers or chemical species having various concentrations in the control channel.

(b) of FIG. 4 shows experimental data on a radial concentration gradient generated by a single source surrounded by the sink. In the present experiment, the source includes 100 μm fluorescein isothiocyanate (FITC) in a phosphate-buffered saline (PBS), and the sink include 1X PBS in which FITC is not contained. The fluorescence intensity of FITC starts from the source and shows a radial gradient. Referring to a graph, the gradient increases rapidly to 10 minutes and represents uniformity after 20 minutes. Although there is no great difference in the concentration gradient after 20 minutes, there are continuous changes in the concentration of FITC in the domain for a long time. This is caused by diffusion into the inlet/outlet port through the channel in the square domain.

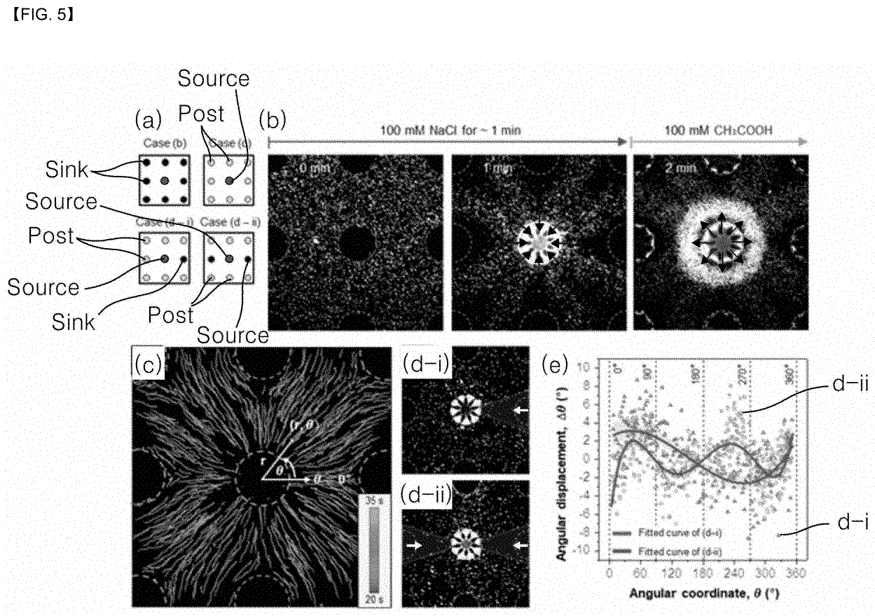

FIG. 5 shows experimental data on dynamic and pixelized control of a source/sink array on a 2D concentration field. The experimental data is obtained by applying 3D-MSA to colloidal transport so as to investigate high efficiency of the selfassembled particle membrane array for active and stable generation of the 2D concentration field. For the pixelized operation of the selfassembled particle membrane array, different solutions flow through the control channel corresponding to each selfassembled particle membrane so that the source and the sink are actively configured. That is, the pixelized control of the source/sink array is configured. For example, the capability of the source for actively converting chemical species into acetic acid from sodium chloride (NaCl) is investigated. First, after 1-μm carboxylated fluorescent particles are loaded into the main channel (the microchannel 1120 of FIG. 1), inlet/outlet ports of the main channel are closed so that the particles are maintained in a stationary state except for displacement by Brown motion. Then, 100 mM NaCl and water flow on the control channel (a channel formed on the control film 1200 of FIG. 1) for about 1 minute with the same configuration as an image represented in (a). In this case, the particles show immediate movement and concentration toward a central source of the main channel. Some particles near the surface of the main channel have opposite transport induced by diffusion-osmosis (DO). However, because DO has a significant effect only on particles close to a wall, most particles are attracted towards a NaCl source on average. A region in which there are no nine particles between the source and the sink, a comparatively sharp local gradient is generated by a source-sink pair. When a NaCl solution is changed into 100 mM acetic acid, the concentrated particles move away from the source. That is, there is a difference between pulling and repulsing depending on the type of the source.

(c) to (d) show a control experiment for setting various source/sink configurations that may generate a different trajectory of colloidal transport in a 2D space. The center of the captured image is set as a pole point of a pole coordinate. In the case of (c), when only a central selfassembled particle membrane is made as a source having attraction and the remaining particle membranes are made as not a sink but a post, the path line of the carboxylated fluorescent particles are tracked. According to this, unlike in eight sinks represented in (b), there is no local gradient that can be made as a source-sink pair. Thus, there is no region in which no particles are present, between the source and the post. Similarly, in the case of one source and one sink pair and one source and two sink pair, the local gradient may be observed. (e) shows experimental results in which each displacement of diffusion particles starting to move for 130 seconds at a distance 240 μm in a radial direction so as to more quantitively analyze and check changes in a trajectory due to the configuration of pairs about two cases represented in (d).

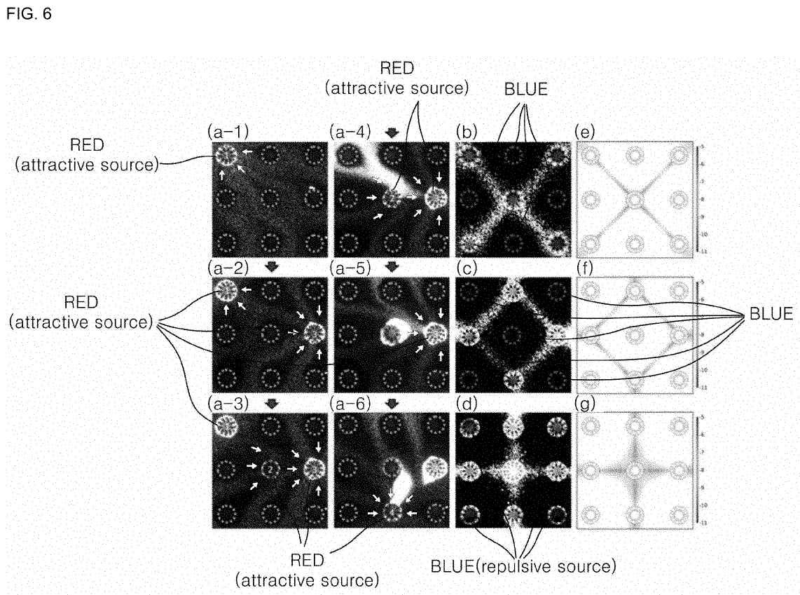

FIG. 6 shows experimental data on spatiotemporal programmability of concentrated field control. For example, according to the operation principle of pixel control described above, a sink is replaced with a source having attraction or vice versa is sequentially replaced so that the particles may be moved to a desired pixel position and concentrated. According to the present experiment, whenever the selfassembled particle membrane is converted into the source having attraction, all conversions cause changes in the shape of a region in which no particles highlighted by red arrows are present. That is, in the changes in the shape, the direction of the local gradient may be finely manufactured and modified by a source-sink pair. Also, colloidal patterns may be generated with the symmetric configuration of several repulsion sources and sinks.

Hereinafter, a method of fabricating the apparatus for generating the microfluidic concentration field shown in FIG. 1 will be described.

First, a microfluidic film 1100 including a base film 1110, a microchannel 1120 through which a fluid flows, and a through passage 1130 that communicates the microchannel 1120 and passes through the base film 1110, is prepared. The microfluidic film 1100 is disposed on a substrate 1300. Next, a selfassembled particle membrane (SAPM) through which the fluid flowing on the microchannel 1120 and the through passage 1130 selectively passes through the SAPM through which the microchannel 1120 and the through passage 1130 communicate with each other, is formed.

Next, a control film through which the fluid may flow and which includes a control channel formed to communicate with the through passage 1130 of the base film 1110, is prepared. The base film and the control film are stacked so that the through passage 1130 and the control channel may communicate with each other.

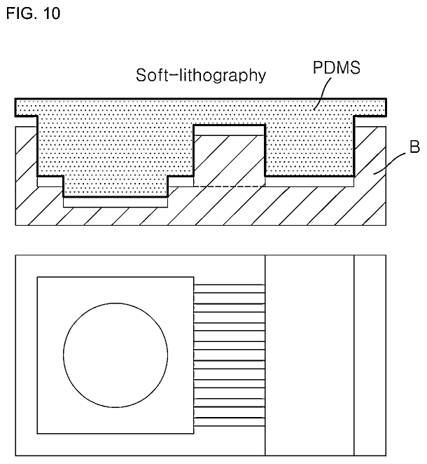

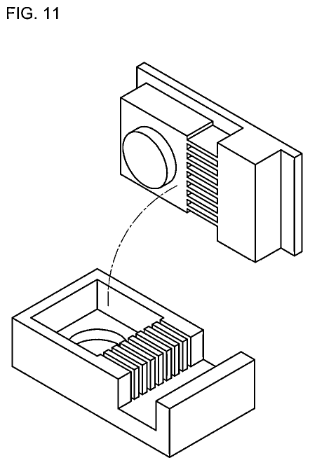

Hereinafter, a process of fabricating the microfluidic film 1100 of FIG. 1 will be described with reference to FIGS. 7 through 14.

The method of fabricating the microfluidic film 1100 includes fabricating a basic mold B (S100), fabricating a master mold by using the basic mold B (S200), and fabricating a microfluidic film by using the master mold (S300). The fabricating of the basic mold B (S100) is a process of fabricating a mold for fabricating the master mold. In the present embodiment, in the fabricating of the basic mold B (S100), the basic mold B is fabricated using a photolithography process. The basic mold B includes a base member formed of a silicon wafer, and the base member includes a first base groove having a storage space formed therein, a second base groove being spaced apart from the first base groove and having a storage space formed therein, and a third base groove through which the first base groove and the second base groove communicate with each other. In the present embodiment, the first base groove includes a first base lower groove having a small width of a lower part, and a first base upper groove that communicates with an upper part of the first base lower groove and extends upward.

Although it will be described below, the first base groove has a structure for forming the through passage 1130 of the microfluidic film 1100. The second base groove has a structure for forming the microchannel 1120 of the microfluidic film 1100. The third base groove has a structure for forming the connection channel 1140 of the microfluidic film 1100.

The fabricating of the basic mold B (S100) undergoes a first exposure operation in which a first photoresist is applied onto the silicon wafer and a first mask having a first pattern for forming the third base groove formed thereon is disposed at an upper portion of the first photoresist and then light is irradiated onto the first mask. Next, the fabricating of the basic mold B (S100) undergoes a first etching operation in which the silicon wafer that has undergone the first exposure operation is etched by using a developing agent. In the present embodiment, the first photoresist is a SU-8 photoresist.

Next, a second exposure operation in which, after the first photoresist is removed, a second photoresist is applied onto the silicon wafer, a second mask having a second pattern for forming the first base lower groove formed thereon is disposed at an upper portion of the second photoresist and then light is irradiated onto the second mask, is performed. A second etching operation in which the silicon wafer that has undergone the second exposure operation is etched by using the developing agent, is performed.

Next, a third exposure operation in which, after the second photoresist is removed, a third photoresist is applied onto the silicon wafer, a third mask having a third pattern for forming the first base upper groove formed thereon and a fourth pattern for forming the second base groove formed thereon is disposed at an upper portion of the third photoresist and then light is irradiated onto the third mask, is performed. A third etching operation in which the silicon wafer that has undergone the third exposure operation is etched by using the developing agent, is performed.

In the silicon wafer according to the present embodiment, the vertical length of the first base upper groove and the vertical length of the second base groove are the same. However, the vertical length of the first base lower groove is smaller than the vertical length of the first base upper groove and the vertical length of the second base groove. In detail, the vertical length of the first base upper groove and the vertical length of the second base groove are the same, 60 μm, and the vertical length of the first base lower groove is formed to be 20 μm.

The width of the cross-section of the first base lower groove is smaller than the width of the cross-section of the first base upper groove. Thus, the first base lower groove and the first base upper groove form a step height. This serves to help the master mold from being easily separated from the basic mold B when the master mold is fabricated through a soft-lithography process by using the basic mold B.

In fabricating of the master mold (S200), the master mold is fabricated by using the basic mold B as a template. In the present embodiment, the master mold formed of polydimethylsiloxane (PDMS) is fabricated by using the soft-lithography process. That is, in the present embodiment, PDMS in a liquid state is injected into the basic mold B and then is cured so that the master mold is fabricated. Any type of polymer for fabricating the master mold may be changed.

Because the master mold is complementarily coupled to the basic mold B, a first protrusion is formed in a portion corresponding to the first base groove, a second protrusion is formed in a portion corresponding to the second base groove, and a groove is formed in a portion corresponding to the third base groove. In particular, the second protrusion has a structure in which widths in a vertical direction are the same. However, a portion of the first protrusion corresponding to the first base lower groove has a small width, and a portion of the first protrusion corresponding to the first base upper grove has a large width.

In the present embodiment, the master mold is formed of a material having higher rigidity than that of the basic mold B. Thus, the mater mold may be repeatedly used, unlike in the basic mold B. Because, in the method of fabricating the microfluidic film according to the present embodiment, cost may be reduced, and mass production is possible compared to a case where the microfluidic film is directly fabricated in a way to fabricate the basic mold B by using the silicon wafer.

The fabricating of the microfluidic film (S300) is a process in which the microfluidic film 1100 including the microchannel 1120 and the through passage 1130 is fabricated by using the master mold as a template. First, the master mold is surface-modified with perfluorooctyltrimethoxysilane (PFOCTS). Next, a glass substrate for forming a template for manufacturing the microfluidic film 1100 is prepared together with the master mold. In this case, the master mold is well attached to the glass substrate, and polyvinyl alcohol (PVA) that is soluble in water is spin-coated.

Next, the master mold is attached onto the glass substrate so that the protruding lower portion of the master mold faces the glass substrate coated with PVA. Next, an OSTEmer resin is loaded between the master mold and the glass substrate. Subsequently, the OSTEmer resin is cured with ultraviolet light (UV) 312 nm. In this case, a curing process by UV makes the OSTEmer resin hard but soft.

Next, the master mold that is reusable is removed. In a state in which the master mold is removed, the cured OSTEmer resin is baked at 80° C. By removing the glass substrate and PVA, the microfluidic film 1100 is fabricated.

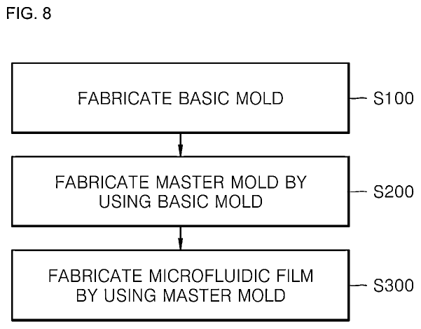

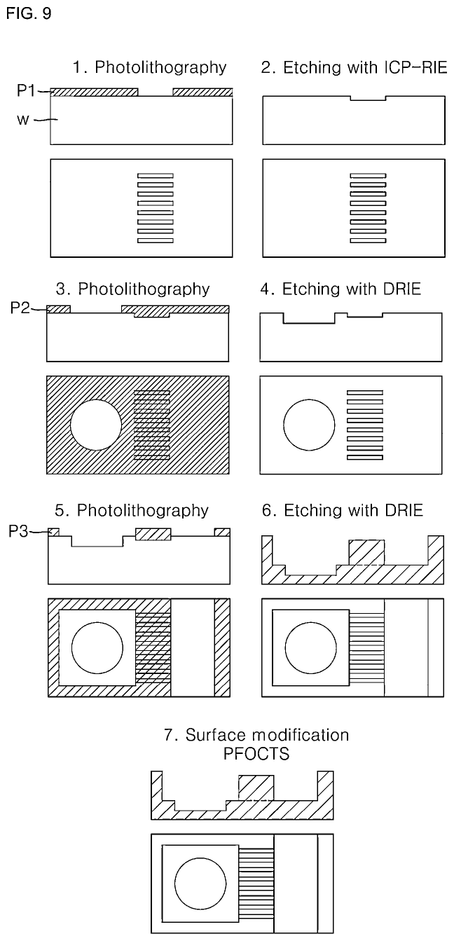

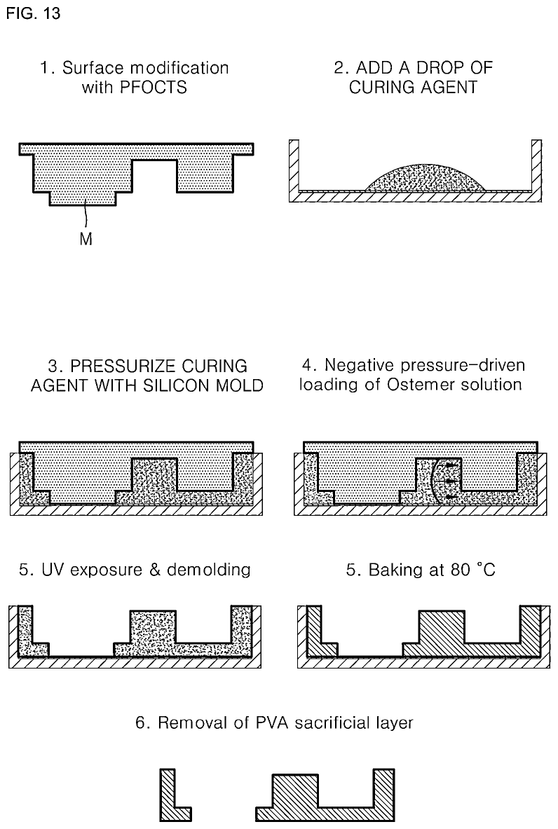

Referring to FIG. 13, a method of fabricating a microfluidic film according to another embodiment of the present invention is shown. The method of fabricating the microfluidic film according to the present embodiment is different from the method of fabricating the microfluidic film shown in FIGS. 8 through 11 that the microfluidic film is fabricated by using not the master mold of PDMS but the master mold of silicon. That is, after the master mold formed of silicon is fabricated by using the basic mold B made in FIG. 9, the microfluidic film is fabricated by using the master mold of silicon. Other procedures are similar to those of the method of fabricating the microfluidic film shown in FIGS. 8 through 11 and thus, a detailed description thereof is omitted.

First, the master mold is surface-modified with PFOCTS. Next, a glass substrate for forming a template for fabricating the microfluidic film 1100 is prepared together with the master mold. In this case, the master mold is well attached to the glass substrate, and the glass substrate is spin-coated with PVA that is soluble in water. Then, a drop of curing agent is added to the glass substrate coated with PVA.

Next, the curing agent is pressurized with a portion of the master mold having an uneven structure (protrusions and grooves) that may be complementarily coupled to the master mold so that the curing agent is uniformly formed between the glass substrate and the master mold. Next, the OSTEmer resin is loaded between the master mold and the glass substrate. Subsequently, the OSTEmer resin is cured with UV 312 nm. In this case, the curing process by UV makes the OSTEmer resin hard but soft.

Next, the master mold is removed. In a state in which the master mold is removed, the cured OSTEmer resin is baked at 80° C. Then, by removing the glass substrate and PVA, the microfluidic film 1100 is fabricated.

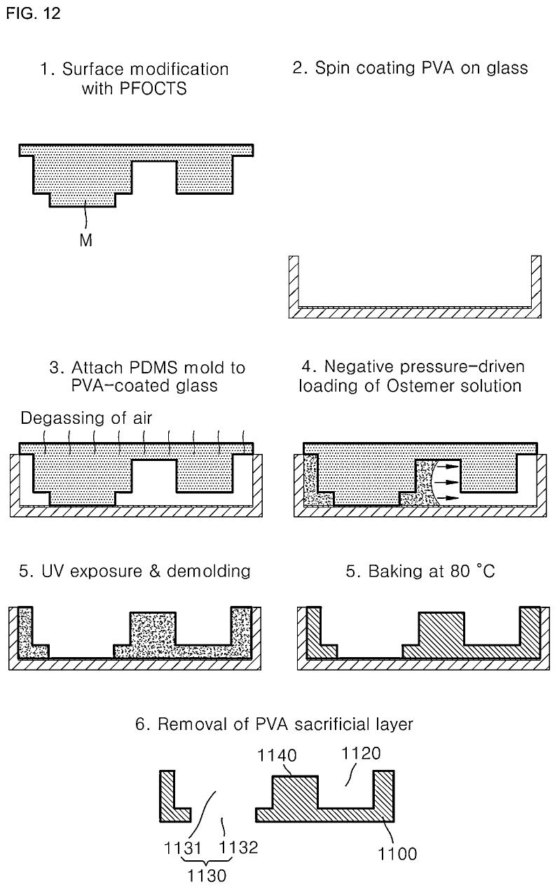

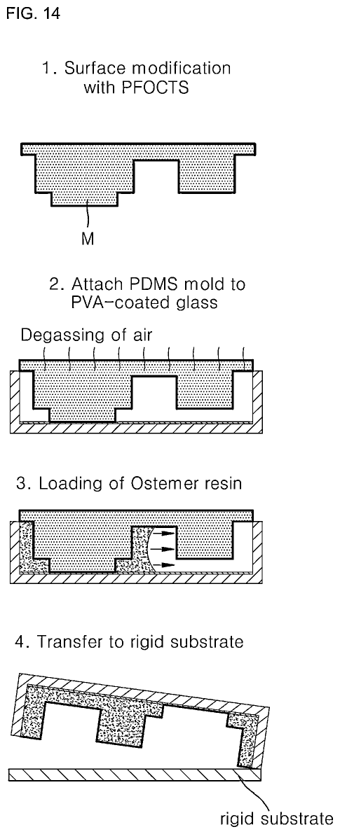

Referring to FIG. 14, a process in which the microfluidic film 1100 fabricated according to FIG. 12 is disposed on a hard substrate, is illustrated. This process is a process in which the microfluidic film 1100 is attached to the hard substrate to fabricate a two-dimensional fluidic module. In this case, the hard substrate is formed of a material having higher rigidity than that of the microfluidic film 1100.

Hereinafter, a method of forming a SAPM on the microfluidic film 1100 will be described.

First, an aqueous particle suspension and silica nanoparticles are injected into a portion of the connection channel 1140 through which the microchannel 1120 and the through passage 1130 communicate with each other. In this case, a portion where the microchannel 1120 and the through passage 1130 communicate with each other, forms a gap that is shallower than the vertical width of the microchannel 1120 upward from the substrate 1100. This is because, when drying with a drying gas to be described below, when the width of the gap portion is large, the aqueous particle suspension and the silica nanoparticles may overflow in a direction of the microchannel 1120 and the membrane is difficult to be easily fixed.

The aqueous particle suspension and the silica nanoparticles are also first injected into the through passage 1130. Next, a drying nitrogen gas (N₂) is blown into a portion where the microchannel 1120 and the through passage 1130 communicate with each other, through the microchannel 1120. Then, the microfluidic film 1000 is dried to remove moisture to form a selfassembled particle membrane.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. An apparatus for generating a microfluidic concentration field, the apparatus comprising:
a substrate;
a base film disposed on the substrate;
a microchannel, which is formed in a space between the substrate and the base film and through which a fluid flows;
a through passage, which communicates with the microchannel and is configured to pass through the base film; and
a membrane, which is formed at a portion where the microchannel and the through passage communicate with each other and allows the fluid flowing along the microchannel and the through passage or a material flowing together with the fluid to selectively pass through the membrane,
wherein a concentration field is formed between the fluid of the through passage and the fluid of the microchannel by the membrane.

2. The apparatus of claim 1, wherein the through passage comprises:
a first through passage formed on an upper portion of the through passage; and
a second through passage having an upper portion communicating with a lower portion of the first through passage and a width that is greater than a width of the first through passage.

3. The apparatus of claim 2, wherein the second through passage is open in a direction of the microchannel from a lower portion of a side surface to a portion spaced apart from the lower portion of the side surface by a set length in an upward direction and communicates with the microchannel.

4. The apparatus of claim 3, wherein a cross-section of the open portion of the second through passage is circular.

5. The apparatus of claim 3, wherein the second through passage communicates with a plurality of microchannels formed in a direction crossing a direction in which the through passage is formed.

6. The apparatus of claim 1, wherein a plurality of through passages are spaced apart from each other.

7. The apparatus of claim 1, further comprising a control film stacked on an upper portion of the base film and communicating with the through passage, wherein the concentration field formed on the microchannel is controlled by the fluid flowing into the base film through a control channel formed on the control film to communicate with the through passage.

8. The apparatus of claim 7, wherein the control channel is formed at a position corresponding to the through passage formed on the base film.

9. A method of fabricating an apparatus for generating a microfluidic concentration field, the method comprising:
preparing a microfluidic film and disposing the microfluidic film on a substrate, the microfluidic film comprising a base film, a microchannel, which is formed on the base film and through which a fluid flows and a through passage communicating with the microfluidic channel and being configured to pass through the base film; and
forming a membrane, the membrane being formed at a portion where the microchannel and the through passage communicate with each other and allowing the fluid flowing along the microchannel and the through passage to selectively pass through the membrane.

10. The method of claim 9, further comprising a coupling operation in which a control film including a control channel through which the fluid flows and communicating with a through passage of the base film is prepared and the base film and the control film are stacked so that the through passage and the control channel communicate with each other.

11. The method of claim 10, wherein the preparing of the microfluidic film and disposing of the microfluidic film on the substrate comprises:
fabricating a basic mold, the basic mold comprising a base member, a first base
groove formed on the base member to extend in a longitudinal direction and having a storage space therein, a second base groove formed on the base member, being spaced apart from the first base groove and having a storage space therein, and a third base groove formed between the first base groove and the second base groove so that the first base groove and the second base groove communicate with each other;
fabricating a master mold that is repeatedly usable by using the basic mold as a template; and
fabricating a microfluidic film by using the master mold as a template, the microfluidic film comprising a microchannel through which the fluid flows and a through passage for communicating with the microfluidic film stacked on an upper portion of the microchannel.

12. The method of claim 11, wherein, in the fabricating of the basic mold, the basic mold is fabricated using a photolithography process.

13. The method of claim 11, wherein, in the fabricating of the master mold, polymer is injected into the basic mold and is cured to fabricate the master mold.

14. The method of claim 10, wherein the through passage comprises:
a first through passage having an upper portion communicating with the control film; and
a second through passage having an upper portion communicating with a lower portion of the first through passage and having a width that is greater than a width of the first through passage, and
the second through passage is open in a direction of the microchannel from a lower portion of a side surface to a portion spaced apart from the lower portion of the side surface by a set length in an upward direction and communicates with the microchannel.

15. The method of claim 9, wherein the forming of the membrane comprises forming a selfassembled particle membrane (SAPM) in which microparticles are stacked and arranged at a portion in which the microchannel and the through passage communicate with each other, by self-assembling.

16. The method of claim 15, wherein the forming of the SAPM comprises:
injecting an aqueous particle suspension and silica nanoparticles into a portion
where the microchannel and the through passage communicate with each other;
blowing a drying nitrogen gas (N₂) into a portion where the microchannel and the through passage communicate with each other, through the microchannel; and
drying the microfluidic film.

17. An apparatus for fluid flow comprising:
a substrate;
a base film disposed on the substrate;
a microchannel, which is defined by a space between the substrate and the base
film and through which a fluid flows;
a through passage, which communicates with the microchannel and is configured
to pass through the base film; and
a membrane, which is formed at a portion where the microchannel and the through passage communicate with each other and allows the fluid flowing along the microchannel and the through passage or a material flowing together with the fluid to selectively pass through the membrane.
